(19) United States
(12) Patent Application Publication
(10) Pub. No.: US 2025/0332447 A1
(43) Pub. Date: Oct. 30, 2025

(54) SYSTEM AND METHOD FOR PROTON THERAPY TREATMENT PLANNING WITH PROTON ENERGY AND SPOT OPTIMIZATION

(71) Applicant: Siemens Healthineers International AG, Steinhausen (CH)

(72) Inventors: Timo KOPONEN, Espoo (FI); Perttu NIEMELA, Espoo (FI)

(73) Assignee: Siemens Healthineers International AG, Steinhausen (CH)

(21) Appl. No.: 19/263,828

(22) Filed: Jul. 9, 2025

Related U.S. Application Data
(62) Division of application No. 16/838,940, filed on Apr. 2, 2020, now Pat. No. 12,390,662.

(51) Int. Cl. A61N 5/10; G16H 20/40

(52) U.S. Cl. CPC A61N 5/1031; A61N 5/1081; G16H 20/40; A61N 2005/1087

(57) ABSTRACT

Methods and systems for proton therapy planning include proton energy and spot optimization that discretizes layers and spots using an optimization algorithm to produce an optimal distribution of layer energies and spots with a relatively smooth dose distribution. The treatment planning algorithms disclosed herein can freely choose the number of spots and the energy levels of the spots. In this way, each spot can be treated as its own layer and is not constrained by the requirements of other spots/layers. Thereafter, the spots defined by the algorithm can be sorted in a list according to energy levels/depth, and the spots can be grouped into blocks according to intensity and location. The blocks can be assigned energy levels based on the corresponding spots, such as an average of all the spots associated with the block. The blocks then are used as the energy layers applied by the proton therapy treatment system.

### CROSS-REFERENCE TO RELATED APPLICATION(S)

This application is a Divisional of U.S. application Ser. No. 16/838,940, filed on Apr. 2, 2020, the entire contents of each of which are incorporated herein by reference.

### FIELD

Embodiments of the present invention generally relate to the field of radiotherapy treatment and systems for same. More specifically, embodiments of the present invention relate to systems and methods for proton therapy treatment planning.

### BACKGROUND

Particle therapy with protons or other ions is a type of radiotherapy that uses an external beam to provide targeted ionizing radiation to a tumor. Protons or other positively charged ions are sent to an accelerator to bring the particles' energy to a predetermined value. The protons or other ions then move through a beam-transport system, where magnets are used to shape, focus and/or direct the proton beam or other ion beam as necessary.

Standard radiation therapy systems deposit energy in “spots” along the path of the beam to a target tumor. However, the reach of the energy also extends beyond the target tumor, and may deliver radiation to healthy tissue around the tumor site. This excess radiation may damage normal tissue or organs near the target area. Moreover, the selection of specific energies and the number of spots are decided based only on patient geometry and hardware constraints. The subsequent optimization to achieve the dosimetric criteria for treatment is traditionally performed only on spot intensities, which can produce less than optimal results. It is appreciated that the term spot intensity is used here as a synonym to monitor unit count, e.g., the intensity is a relative quantity proportional to the number of protons per spot, as delivered by the treatment machine.

Radiation treatment plans can be optimized according to given dose volume objectives for target volume and organs at risk and according to plan robustness using commercially available treatment planning systems. Dose distributions are calculated using beam characteristics and a machine specific dose calibration. However, machine or system limitations can lead to a translation of an aimed dose distribution into machine/treatment delivery system parameters that may generate an unacceptable treatment plan. Additionally, the generated treatment plan may not use the full system/machine capability to make use of the system in the most efficient and reliable manner. The treatment plan can be optimized for efficiency and may employ a trial-and-error modification of several complex associated plan parameters (e.g., energy layer distance, spot size or spot spacing) required for multi-directional optimization. Even if an optimized treatment plan finally passes the criteria for plan quality and treatment delivery time, the manual trial and-error-process is time consuming and may not be able to achieve the optimal delivery efficiency.

More specifically, particle therapy planning using existing techniques is constrained by the delivery of doses in discrete, predetermined increments. For example, to deliver dose to a 3D volume, multiple discrete beam energies are required and further, the dose with energy must be delivered in discrete spots. Traditionally, the discretization scheme, including the selection of specific energies, the number of spots, and their locations, are decided based on patient geometry and hardware constraints, and the treatment planning optimization is limited by these constraints. Rather, the subsequent optimization to achieve the dosimetric criteria for treatment is traditionally performed only on spot intensities. Because there is a lower limit for the intensity per each spot, the layers used to deliver the dose will only include a relatively low number of spots. This often disadvantageously results in an uneven dose distribution, which is considered clinically suboptimal.

Because layer energies are pre-determined by existing treatment planning solutions, there is nothing the optimization algorithm can do to resolve these constraints. Therefore, what is needed is an approach to particle therapy treatment that discretizes layers and spots using an optimization algorithm without the existing constraints, that produces an optimal distribution of layer energies and spots with a smoother (e.g., more even) dose distribution, and that can advantageously produce fewer layers and potentially fewer spots, thereby reducing the time taken to treat a patient. Therefore, what is needed is an approach to particle therapy treatment that addresses these issues.

### SUMMARY

More specifically, what is needed is an approach to particle therapy treatment that discretizes layers and spots using an optimization algorithm without the existing constraints, that produces an optimal distribution of layer energies and spots with a smoother (e.g., more even) dose distribution, and that can advantageously produce fewer layers and potentially fewer spots, thereby reducing the time required to treat a patient. Accordingly, methods and systems for proton therapy planning that include proton energy and spot optimization that discretizes layers and spots using an optimization algorithm to produce an optimal distribution of layer energies and spots with a relatively smooth dose distribution are disclosed herein. Embodiments of the present invention can thereby produce fewer layers and potentially fewer spots, thereby advantageously reducing the time required to treat a patient.

According to one embodiment, a system for proton therapy treatment is disclosed. The system includes a gantry including a nozzle emitting a controllable proton beam, a proton therapy treatment system that controls the gantry according to a treatment plan, and a treatment planning system including a memory, and a processor operable to perform a method for generating the treatment plan. The method includes receiving treatment planning parameters, determining a number of spots and energy levels of the spots according to the treatment planning parameters, calculating intensities for the spots according to the energy levels, discretizing the spots into a plurality of blocks according to a location of the spots and the intensities of the spots, wherein the treatment plan

comprises the blocks, and where the block represent an energy layer applied by the gantry, and outputting the treatment plan.

According to some embodiments, the method includes treating a patient using the gantry according to the treatment plan.

According to some embodiments, the discretizing the spots into a plurality of blocks is performed iteratively until a clinical goal is achieved.

According to some embodiments, the method includes determining a plurality of angles for performing proton therapy treatment and where the determining a number of spots and energy levels of the spots is further based on the plurality of angles.

According to some embodiments, the method includes receiving the number of layers as user input, where the determining a number of spots and energy levels of the spots is further based on the number of layers.

According to some embodiments, the method includes sorting the spots according to depth before discretizing the spots.

According to some embodiments, the method includes sorting the spots according to energy level before discretizing the spots.

According to some embodiments, the energy layers have a fixed width.

According to some embodiments, the method includes the energy layers have a fixed intensity.

According to another embodiment, a method for proton therapy treatment is disclosed. The method includes receiving treatment planning parameters, determining a number of spots and energy levels of the spots according to the treatment planning parameters, sorting the spots based on a depth of a respective spot, calculating intensities for the spots according to the energy levels, discretizing the spots into a plurality of blocks according to a location of the spots and the intensities of the spots, where each block represents an energy layer applied by the gantry, and outputting the treatment plan, wherein the treatment plan comprises the blocks.

According to a different embodiment, a non-transitory computer-readable storage medium having embedded therein program instructions, which when executed by one or more processors of a device, causes the device to execute a method for proton therapy treatment. The method includes receiving treatment planning parameters, determining a number of spots and energy levels of the spots according to the treatment planning parameters, sorting the spots based on a depth of a respective spot, calculating intensities for the spots according to the energy levels, discretizing the spots into a plurality of blocks according to a location of the spots and the intensities of the spots, where each block represents an energy layer applied by the gantry, and outputting the treatment plan, wherein the treatment plan comprises the blocks.

### BRIEF DESCRIPTION OF THE DRAWINGS

The accompanying drawings, which are incorporated in and form a part of this specification, illustrate embodiments of the invention and, together with the description, serve to explain the principles of the invention:

FIG. 1 shows a block diagram of an example of a computing system upon which the embodiments described herein may be implemented.

FIG. 2 is a block diagram showing selected components of a radiation treatment system upon which embodiments according to the present invention can be implemented.

FIG. 3 illustrates elements of a radiation treatment system in accordance with embodiments according to the invention.

FIG. 4 is a block diagram illustrating components in a process for creating an optimized proton therapy treatment plan in embodiments according to the present invention.

FIG. 5 is a flow-chart depicting an exemplary sequence steps for automatically creating an optimized proton therapy treatment plan according to embodiments of the present invention.

FIG. 6 is a diagram of an exemplary proton therapy treatment plan with predetermined energy layers.

FIG. 7 is a diagram of an exemplary proton therapy treatment plan optimized according to a spot distribution using customized energy layers having a fixed intensity per layer.

### DETAILED DESCRIPTION

Reference will now be made in detail to several embodiments. While the subject matter will be described in conjunction with the alternative embodiments, it will be understood that they are not intended to limit the claimed subject matter to these embodiments. On the contrary, the claimed subject matter is intended to cover alternative, modifications, and equivalents, which may be included within the spirit and scope of the claimed subject matter as defined by the appended claims.

Furthermore, in the following detailed description, numerous specific details are set forth in order to provide a thorough understanding of the claimed subject matter.

However, it will be recognized by one skilled in the art that embodiments may be practiced without these specific details or with equivalents thereof. In other instances, well-known methods, procedures, components, and circuits have not been described in detail as not to unnecessarily obscure aspects and features of the subject matter.

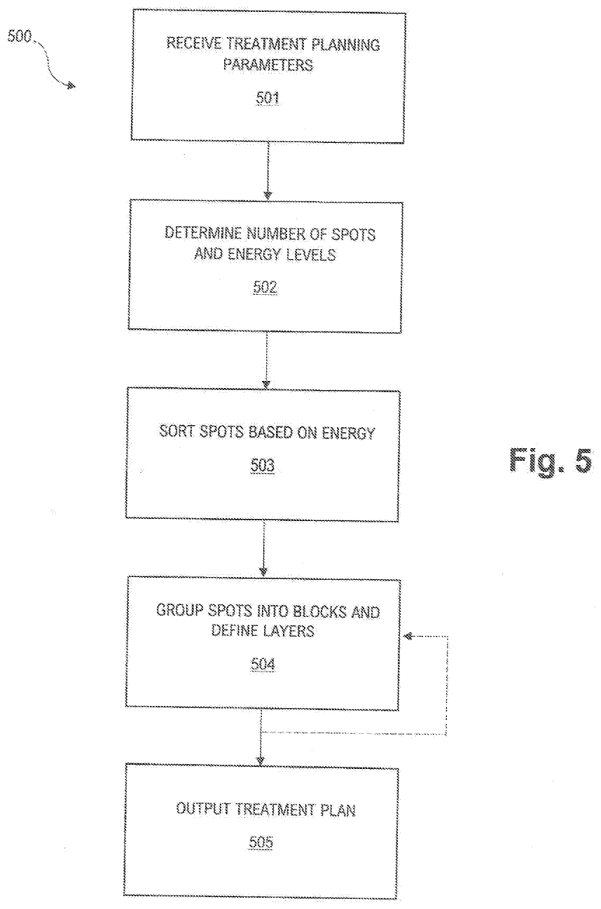

Portions of the detailed description that follows are presented and discussed in terms of a method. Although steps and sequencing thereof are disclosed in a figure herein describing the operations of this method, such steps and sequencing are exemplary. Embodiments are well suited to performing various other steps or variations of the steps recited in the flowchart (e.g., FIG. 5) of the figures herein, and in a sequence other than that depicted and described herein.

Some portions of the detailed description are presented in terms of procedures, steps, logic blocks, processing, and other symbolic representations of operations on data bits that can be performed on computer memory. These descriptions and representations are the means used by those skilled in the data processing arts to most effectively convey the substance of their work to others skilled in the art. A procedure, computer-executed step, logic block, process, etc., is here, and generally, conceived to be a self-consistent sequence of steps or instructions leading to a desired result. The steps are those requiring physical manipulations of physical quantities. Usually, though not necessarily, these quantities take the form of electrical or magnetic signals capable of being stored, transferred, combined, compared, and otherwise manipulated in a computer system. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the following discussions, it is appreciated that throughout, discussions utilizing terms such as “generating,” “writing,” “including,” “storing,” “transmitting,” “traversing,” “associating,” “identifying,” “optimizing” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

Some embodiments may be described in the general context of computer-executable instructions, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

### Energy and Spot Optimization for Proton Therapy Treatment

The following description is presented to enable a person skilled in the art to make and use the embodiments of this invention; it is presented in the context of a particular application and its requirements. Various modifications to the disclosed embodiments will be readily apparent to those skilled in the art, and the general principles defined herein may be applied to other embodiments and applications without departing from the spirit and scope of the present disclosure. Thus, the present invention is not limited to the embodiments shown, but is to be accorded the widest scope consistent with the principles and features disclosed herein.

Methods and systems for proton therapy planning including proton energy and spot optimization that discretizes layers and spots using an optimization process are described herein. The optimization process produces an optimal distribution of layer energies and spots with a relatively smooth dose distribution, and can freely choose the number of spots and the energy levels of the spots. In this way, each spot can be treated as its own layer and is not constrained by the requirements of other spots/layers. Thereafter, the spots defined by the process can be sorted into a list according to energy levels/depths, and the spots can be grouped into blocks. The blocks can be assigned energy levels based on the corresponding spots, for example, using an average of all of the spots associated with the block. The blocks are then used to define the energy levels applied by the proton therapy treatment system.

Embodiments of the present invention can thereby produce fewer layers and potentially fewer spots, thereby advantageously reducing the time required to treat a patient. The optimization processes, e.g., algorithms, described herein can split individual spots among different layers to avoid creating spots that are below the minimum energy threshold deliverable by the treatment system. The spots can be divided into layers such that the layers receive substantially equal intensities. For example, the spots can be divided into layers so that each layer receives a substantially equal number of protons delivered by the proton therapy treatment system. In general, a treatment plan that is optimized using fewer layers leads to a faster delivery time. On the other hand, embodiments of the present invention can also optimize proton treatment plans for accuracy by using more spots and/or layers to better fit the target volume. The spots can be distributed at fixed intervals (e.g., 3 mm spacing) or distributed based on variations such as beam energy and/or beam range. In all cases, the general principle holds that the intensity of a layer is the sum of the intensities of all spots on a given layer.

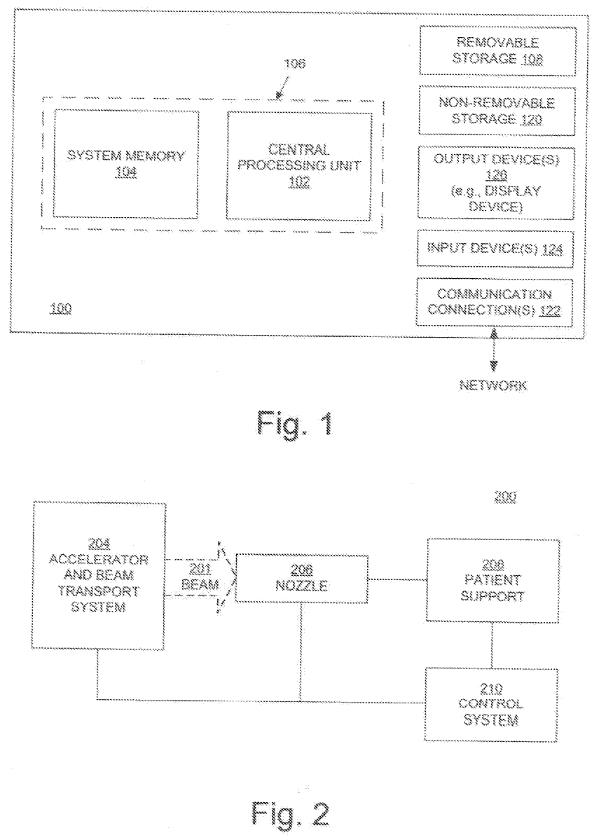

FIG. 1 shows a block diagram of an example of a computing system 100 upon which the embodiments described herein may be implemented. In a basic configuration, the system 100 includes at least one processing unit 102 and a memory 104. This most basic configuration is illustrated in FIG. 1 by dashed line 106. The system 100 may also have additional features and/or functionality. For example, the system 100 may also include additional storage (removable and/or non-removable) including, but not limited to, magnetic or optical disks or tape. Such additional storage is illustrated in FIG. 1 by removable storage 108 and non-removable storage 120. The system 100 may also contain communications connection(s) 122 that allow the device to communicate with other devices, e.g., in a networked environment using logical connections to one or more remote computers.

The system 100 of FIG. 1 also includes input device(s) 124 such as keyboard, mouse, pen, voice input device, touch input device, etc. Output device(s) 126 such as a display device, speakers, printer, etc., are also included.

In the example of FIG. 1, the memory 104 includes computer-readable instructions, data structures, program modules, and the like. Depending on how it is to be used, the system 100—by executing the appropriate instructions or the like—can be used to implement a planning system used that discretizes layers and spots using an optimization algorithm (comprising computer readable instructions) to produce an optimal distribution of layer energies and spots with a relatively smooth dose distribution. The treatment planning algorithms executed by CPU 102 can freely choose the number of spots and the energy levels of the spots so that each spot can be treated as its own layer unconstrained by the requirements of other spots/layers. Thereafter, the spots defined by the algorithm can be sorted in a list according to energy levels/depth using CPU 102, and the spots can be grouped into blocks. The blocks can be assigned energy levels based on corresponding spots, for example, using an average of all spots associated with the block. More generally, system 100 can be used to generate and/or optimize proton therapy treatment plans in accordance with the present invention.

FIG. 2 is a block diagram showing selected components of a radiation treatment system 200 upon which embodiments according to the present invention can be implemented. In the example of FIG. 2, the system 200 includes an accelerator and beam transport system 204 that generate and/or accelerate a beam 201. Embodiments according to the invention can generate and deliver beams of various types including, for instance, proton beams, electron beams, neutron beams, photon beams, ion beams, or atomic nuclei beams (e.g., using elements such as carbon, helium, or lithium) and the like. The operations and parameters of the accelerator and beam transport system 204 are controlled so that the intensity, energy, size, and/or shape of the beam are dynamically modulated or controlled during treatment of a patient according to an optimized radiation treatment plan produced by system 100 as discussed above.

A recent radiobiology study has demonstrated the effectiveness of delivering an entire, relatively high therapeutic radiation dose to a target within a single, short period of time. This type of treatment is referred to generally herein as FLASH radiation therapy (FLASH RT). Evidence to date suggests that FLASH RT advantageously spares normal, healthy tissue from damage when that tissue is exposed to only a single irradiation for only a very short period of time. For FLASH RT, the accelerator and beam transport system 204 can generate beams that can deliver at least four (4) grays (Gy) in less than one second, and may deliver as much as 20 Gy or 50 Gy, or more, in less than one second. The control system 210 can execute a treatment plan for FLASH RT, and the plan can be generated or optimized by system 100 executing an optimization algorithm in accordance with embodiments of the present invention.

The nozzle 206 is used to aim the beam toward various locations (a target) within a patient supported on the patient support device 208 (e.g., a chair, couch, or table) located in a treatment room. A target may be an organ, a portion of an organ (e.g., a volume or region within the organ), a tumor, diseased tissue, or a patient outline, for instance.

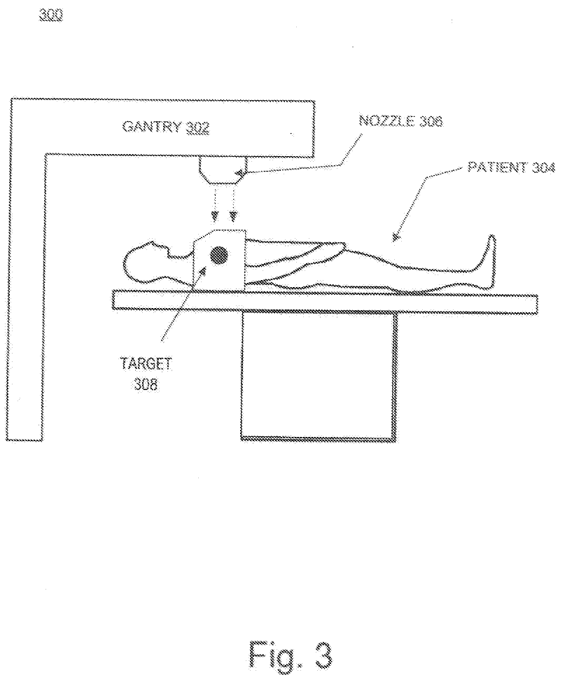

The nozzle 206 may be mounted on or may be a part of a gantry (FIG. 3) that can be moved relative to the patient support device 208, which may also be moveable. In embodiments, the accelerator and beam transport system 204 are also mounted on, or are a part of, the gantry structure; in another embodiment, the accelerator and beam transport system are separate from (but in communication with) the gantry structure.

The control system 210 of FIG. 2 receives and implements a prescribed treatment plan generated and/or optimized according to embodiments of the present invention. In embodiments, the control system 210 includes a computing system having a processor, memory, an input device (e.g., a keyboard), and perhaps optionally a display; the system 100 of FIG. 1 is an example of such a platform for the control system 210. The control system 210 can receive data regarding the operation of the system 200. The control system 210 can control parameters of the accelerator and beam transport system 204, nozzle 206, and patient support device 208, including parameters such as the energy, intensity, size, and/or shape of the beam, direction of the nozzle, and position of the patient support device (and the patient) relative to the nozzle, according to data the control system 210 receives and according to the radiation treatment plan.

FIG. 3 illustrates elements of a radiation treatment system 300 for treating a patient 304 in embodiments according to the present invention. The system 300 is an example of an implementation of the radiation treatment system 200 of FIG. 2, for example. In embodiments, the gantry 302 and nozzle 306 can be moved up and down the length of the patient 304 and/or around the patient, and the gantry and nozzle can move independently of one another. While the patient 304 is supine in the example of FIG. 3, the invention is not so limited to this orientation. For example, the patient 304 can instead be seated in a chair or standing. The gantry 302 can be controlled by a treatment system using an optimized treatment plan generated according to embodiments of the present invention.

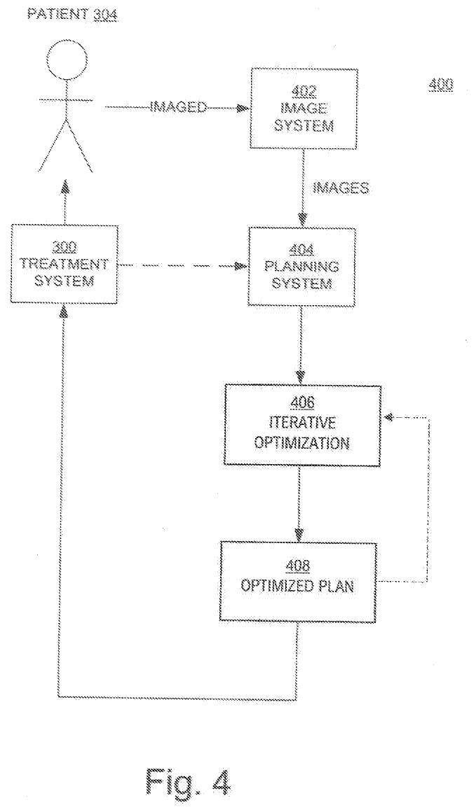

With regard to FIG. 4, an exemplary proton therapy system 400 for imaging and treating a patient 304 is depicted according to embodiments of the present invention. In the example of FIG. 4, patient 304 is imaged using an image system 402 that uses, for example, x-rays, magnetic resonance imaging (MRI), and computed tomography (CT). When CT or MRI imagery, for example, is used, a series of two-dimensional (2D) images are taken from a 3D volume. Each 2D image is an image of a cross-sectional “slice” of the 3D volume. The resulting collection of 2D cross-sectional slices can be combined to create a 3D model or reconstruction of the patient's anatomy (e.g., internal organs). The 3D model will contain organs of interest, which may be referred to as structures of interest herein. Those organs of interest include the organ targeted for radiation therapy (a target), as well as other organs that may be at risk of radiation exposure during treatment. According to some embodiments, the imaging process is a separate process from the treatment planning process, and the treatment planning process can include receiving imaging data from a prior imaging session, for example.

One purpose of the 3D model is the preparation of a radiation treatment plan. To develop a patient-specific radiation treatment plan, information is extracted from the 3D model to determine parameters such as organ shape, organ volume, tumor shape, tumor location in the organ, and the position or orientation of several other structures of interest as they relate to the affected organ and any tumor. The radiation treatment plan can specify, for example, how many radiation beams to use and from which angle each of the beams will be delivered.

In embodiments according to the present invention, the images from the image system 402 are input to a planning system 404. In embodiments, the planning system 404 includes a computing system having a processor, memory, an input device (e.g., a keyboard), and a display. The system 100 of Figure I is an example of a platform for the planning system 404.

Continuing with reference to FIG. 4, the planning system 404 executes software that is capable of producing an optimized treatment plan for treating patient 304. The treatment planning system 404 can receive imagery data generated by image system 402 to determine constraints regarding patient anatomy along with treatment planning parameters, such as other patient-related data and machine parameters. The treatment planning parameters are discretized into spots with defined energy levels, and the spots are sorted according to their depth/energy level. The planning system 404 can optimize the treatment plan according to an optimization algorithm that determines an optimal distribution of layers with a relatively smooth dose distribution. The treatment plan can be optimized in accordance with various prescribed results. For instance, the treatment plan can be optimized to use fewer layers leading to a faster delivery time, or can be optimized for accuracy or 3D dose homogeneity by using more layers to conform to the target volume. The planning system 404 can also receive user input that defines the number of layers used in the optimized plan 408, for example.

The treatment planning system 404 outputs an optimized plan 408 according to an iterative optimizing algorithm 406 as described herein according to embodiments of the present invention. The spots can be distributed at fixed intervals (e.g., 3 mm spacing) or based on variations such as beam energy and/or beam range, and the spots are added to a list of spots that is sorted according to energy level or depth of the spot. Based on the energy levels of the spots and/or the depth/location of a spot, the spots are assigned intensities, where the intensity of a layer is the sum of the intensities of all spots of the layer. The spots are then discretized into blocks according to the energies and intensities, where each block represents a distinct layer. Discretizing the spots into blocks can be an iterative process that is performed repeatedly until a clinical goal or optimization goal is achieved. The optimized plan 408 is then used to configure treatment system 300 for performing proton therapy treatment on patient 304 using gantry 302, for example.

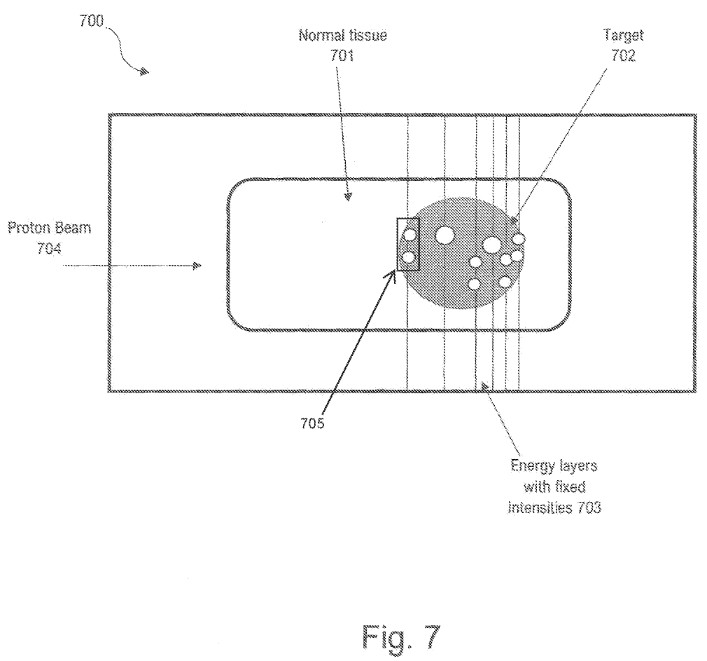

With regard to FIG. 5, an exemplary sequence of computer implemented steps for automatically generating a proton therapy treatment plan is depicted according to embodiments of the present invention. The treatment plan receives treatment planning parameters at step 501, and the parameters may include patient anatomy data and machine parameters. The treatment planning parameters are discretized into spots with defined energy levels at step 502. At this point each spot may have a separate energy level. At step 503, the spots are added to a list of spots and the list is sorted according to energy level or depth of the spot. An optimization algorithm is executed at step 504 to produce an optimal distribution of layer energies and spots with a relatively smooth dose distribution. The spots can be distributed at fixed intervals (e.g., 3 mm spacing) or based on variations such as beam energy and/or beam range, and specifically, the distribution can be based on the energy levels of the spots and/or the depth/location of a spot, the spots are assigned intensities, where the intensity of a layer is the sum of the intensities of all spots of the layer. The spots are then discretized into blocks according to the energies, intensities, and the location of the spots, and each block (705 in FIG. 7) represents a distinct energy layer to be applied by a proton therapy system. Discretizing the spots into blocks can be an iterative process that is performed repeatedly until a clinical goal or optimization goal is achieved.

At step 505, the optimized treatment plan is output, for example, as a computer readable data file. The data file can be stored in memory and used by a proton therapy treatment system to emit a controllable proton beam according to the treatment plan, using a gantry, for example.

According to some embodiments, the optimization algorithm also determines optimal beam angles for a clinical goal or optimization goal and the spots are generated and discretized according to the beam angles.

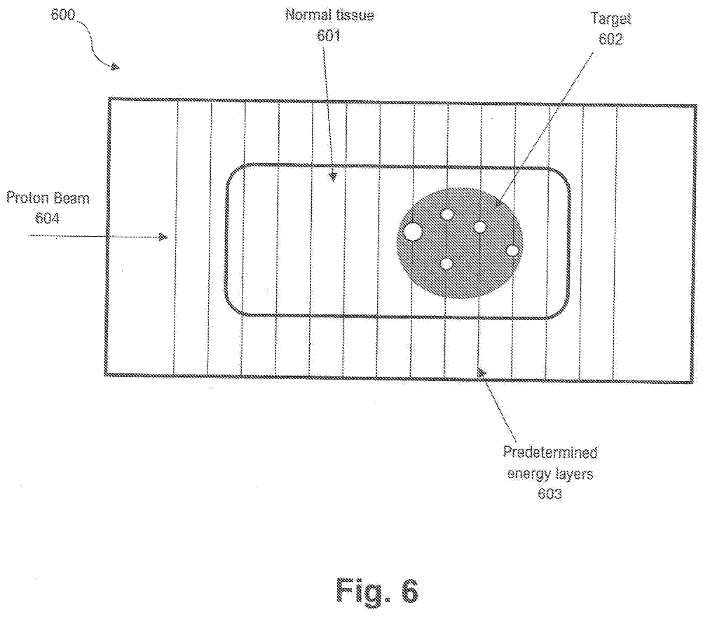

With regard to FIG. 6, an exemplary proton therapy treatment plan 600 applied to a target volume 602 (e.g., a tumor or organ) surrounded by normal tissue 601 for proton therapy treatment is depicted. The treatment plan is generated using limited optimization based on dose volume objectives for the target volume 602 and organs at risk (e.g., normal tissue 601), and machine parameters. Dose distributions are calculated according to the constraints of beam characteristics of proton beam 604 and a machine specific dose calibration. However, the treatment plan depicted in FIG. 6 may not use the full system/machine capability to make use of the system in the most efficient and reliable manner. Moreover, the application of treatment plan 600 at the machine may fail or not achieve the optimal delivery efficiency as requested by plan objectives during treatment planning due to machine specific capability limitations of combined plan parameters that are not taken into account.

As depicted in FIG. 6, the treatment plan is constrained because it is limited to fixed energy layers for discretizing spots produced by proton beam 604 for treating the target volume 602. The energy levels of the predefined layers are defined based on limited considerations of the patient anatomy and characteristics of the treatment device, and a scanning pattern using layers 603 is applied by beam 604 based on these characteristics. Furthermore, using this approach, the deepest layer of the target 602 receives the highest intensity, and layers closer to the surface receive significantly lower intensity, making it difficult to optimize for the surface layers.

Moreover, using the approach depicted in FIG. 6, no less than a certain minimum intensity can be applied to a single spot. Therefore, after optimization, if the intensity of a spot is less than the minimum intensity, the spot must be removed from the plan to generate a plan that is deliverable by the machine. If all spots of an energy layer are below the minimum intensity, the entire layer may need to be removed. A simple removal of spots/layers can change the treatment plan such that the dose distribution is no longer optimal. Moreover, failure to remove these spots or layers from the treatment plan during the planning phase may lead to a failure of the treatment, in which case the treatment must be stopped and restarted after the layer is removed. A more efficient approach involves customizing the number of layers and positioning them as necessary to achieve the clinical objectives, as depicted in FIG. 7 below.

With regard to FIG. 7, an exemplary optimized proton therapy treatment plan 700 applied to a target volume 702 (e.g., a tumor or organ) surrounded by normal tissue 701 for proton therapy treatment is depicted. The treatment plan 700 is initially discretized to layers as if there is infinite number of layers, and an iterative optimization process distributes spots using the layers to achieve the clinical goal of treatment plan 700 and ensures that the plan is actually deliverable by the treatment device (e.g., gantry 302). According to some embodiments, an input number of layers is received as user input (for instance). The optimization algorithm then places the spots in a list sorted by range/energy level, and calculates intensities for each spot (e.g., based on energy level/depth) to determine an resultant (e.g., optimal) distribution of layers for achieving the clinical goal of treatment plan 700 using proton beam 704. As depicted in FIG. 7, the treatment plan 700 includes 6 layers 703, and each layer applies ⅙ of the total intensity (number of protons) of treatment plan 700. In this way, the intensities of the layers are fixed, and the spacing of the layers in unconstrained. Splitting the spots among the layers using the principle of equal layer intensities avoids creating spots and layers that are below the minimum intensity deliverable by the treatment system. Note that the principle of equal layer intensities is used here as an example only and other principles could be used to distribute the spots into energy layers. For example, an arbitrary energy-dependent function could be designed to distribute the intensity of the energy layers unevenly. In general, a treatment plan that is optimized using fewer layers leads to a faster delivery time. Moreover, optimizing the treatment plan for accuracy tends to use more layers to better fit the target volume. The spots can be distributed at fixed intervals (e.g., 3 mm spacing) using proton beam 704, or can be distributed based on variations such as beam energy and/or beam range. In either case, the intensity of a layer is the sum of the intensities of all spots on a given layer.

According to some embodiments, the distribution of the layers and/or the energy levels thereof are manually defined by a user. The user-defined energy levels can include layers with an intensity equal to 0 such that no protons are delivered at certain energies, and these layers can be automatically removed by the treatment planning system after the optimization process. The layers can be defined by the user at fixed distances, or custom distances, for example.

Embodiments of the present invention are thus described. While the present invention has been described in particular embodiments, it should be appreciated that the present invention should not be construed as limited by such embodiments, but rather construed according to the following claims.

What is claimed is:

1. A method for proton therapy treatment via a proton therapy treatment system, the method comprising:
receiving treatment planning parameters;
determining a quantity of spots, among a plurality of spots, according to the treatment planning parameters;
determining an energy for each spot, among the plurality of spots, according to the treatment planning parameters;
calculating intensities for the plurality of spots according to the energy for each spot, among the plurality of spots;
discretizing the plurality of spots into a plurality of blocks according to locations of the plurality of spots and intensities of the plurality of spots, to generate a treatment plan for the proton therapy treatment, wherein
the treatment plan includes the plurality of blocks, and
each block, among the plurality of blocks, represents an energy layer, among a number of energy layers, applied by a controllable proton beam of the proton therapy treatment system; and
outputting the treatment plan to the proton therapy treatment system to perform the proton therapy treatment, wherein
according to the treatment plan, the proton therapy treatment system automatically controls at least one of an intensity, energy, size or shape of the controllable proton beam emitted by the proton therapy treatment system.

2. The method of claim 1, further comprising:
controlling, according to the treatment plan, a gantry structure of the proton therapy treatment system to perform particle therapy on a patient.

3. The method of claim 1, wherein the discretizing the plurality of spots is performed iteratively until a clinical goal is achieved.

4. The method of claim 1, further comprising:
determining a plurality of angles for performing the proton therapy treatment, and wherein
the determining of the quantity of spots and the determining of the energy of each spot is further based on the plurality of angles.

5. The method of claim 1, further comprising:
receiving the number of energy layers as user input, and wherein
the determining of the quantity of spots and the determining of the energy for each spot is further based on the number of energy layers.

6. The method of claim 1, wherein the energy layers are spaced apart from one another by a fixed spacing.

7. The method of claim 1, wherein the energy layers have a fixed total intensity.

8. The method of claim 1, further comprising:
sorting the plurality of spots according to depth before discretizing the plurality of spots.

9. The method of claim 1, further comprising:
sorting the plurality of spots according to the energy of each spot before discretizing the plurality of spots.

10. The method of claim 1, wherein each energy layer, among the number of energy layers, has the same intensity as other energy layers among the number of energy layers.

11. A non-transitory computer-readable storage medium, storing computer-executable instructions that, when executed by at least one processor of a proton therapy treatment system, cause the proton therapy treatment system to perform a method for proton therapy treatment, the method comprising:
receiving treatment planning parameters;
determining a quantity of spots, among a plurality of spots, according to the treatment planning parameters;
determining an energy for each spot, among the plurality of spots, according to the treatment planning parameters;
calculating intensities for the plurality of spots according to the energy for each spot, among the plurality of spots;
discretizing the plurality of spots into a plurality of blocks according to locations of the plurality of spots and intensities of the plurality of spots, to generate a treatment plan for the proton therapy treatment, wherein
the treatment plan includes the plurality of blocks, and
each block, among the plurality of blocks, represents an energy layer, among a number of energy layers, applied by a controllable proton beam of the proton therapy treatment system; and
outputting the treatment plan to the proton therapy treatment system to perform the proton therapy treatment, wherein
according to the treatment plan, the proton therapy treatment system automatically controls at least one of an intensity, energy, size or shape of the controllable proton beam emitted by the proton therapy treatment system.

12. The non-transitory computer-readable storage medium of claim 11, wherein the method further comprises:
controlling, according to the treatment plan, a gantry structure of the proton therapy treatment system to perform particle therapy on a patient.

13. The non-transitory computer-readable storage medium of claim 11, wherein the discretizing the plurality of spots is performed iteratively until a clinical goal is achieved.

14. The non-transitory computer-readable storage medium of claim 11, wherein the method further comprises:
determining a plurality of angles for performing the proton therapy treatment, and wherein
the determining of the quantity of spots and the determining of the energy of each spot is further based on the plurality of angles.

15. The non-transitory computer-readable storage medium of claim 11, wherein the method further comprises:
receiving the number of energy layers as user input, and wherein
the determining of the quantity of spots and the determining of the energy for each spot is further based on the number of energy layers.

16. The non-transitory computer-readable storage medium of claim 11, wherein the energy layers are spaced apart from one another by a fixed spacing.

17. The non-transitory computer-readable storage medium of claim 11, wherein the energy layers have a fixed total intensity.

18. The non-transitory computer-readable storage medium of claim 11, wherein the method further comprises:
sorting the plurality of spots according to depth before discretizing the plurality of spots.

19. The non-transitory computer-readable storage medium of claim 11, wherein the method further comprises:
sorting the plurality of spots according to the energy of each spot before discretizing the plurality of spots.

20. The non-transitory computer-readable storage medium of claim 11, wherein each energy layer, among the number of energy layers, has the same intensity as other energy layers among the number of energy layers.
